(19) United States
(12) Patent Application Publication
(10) Pub. No.: US 2026/0263328 A1
(43) Pub. Date: Sep. 10, 2026

(54) Dental Remineralization Using Catechol-Based Compounds

(71) Applicant: Mussel Polymers, Inc., Bethlehem, PA (US)

(72) Inventors: Eric Anderson, Princeton, NJ (US); Patrick Cunningham, Allentown, PA (US)

(21) Appl. No.: 19/166,514

(22) PCT Filed: Mar. 20, 2024
(86) PCT No.: PCT/US2024/020662

§ 371 Date: Sep. 18, 2025

Related U.S. Application Data
(60) Provisional application No. 63/491,297, filed on Mar. 21, 2023.

(51) Int. Cl. A61K 6/20; A61K 6/62; A61K 6/891

(52) U.S. Cl. CPC A61K 6/20; A61K 6/62; A61K 6/891

(57) ABSTRACT

The present disclosure provides compositions and methods for remineralizing tooth dentin in a subject using one or more catechol-containing material. The present disclosure also provides compositions and methods for treating dental hypersensitivity in a subject comprising contacting exposed dentinal tubules with a catechol-containing material. The present disclosure also provides compositions and methods for restoring acid-etched tooth dentin in a subject comprising contacting the tooth dentin with a catechol-containing material.

### TECHNICAL FIELD

The present invention relates to the field of dental and hard tissue treatment. More specifically, the present invention relates to remineralization of tooth dentin and the catechol-containing materials used for such remineralization.

### CROSS REFERENCE TO RELATED APPLICATIONS

This application claims priority to and the benefit of U.S. Provisional Patent Application No. 63/491,297 (filed Mar. 21, 2023), which is incorporated by reference herein in its entirety for any and all purposes.

### BACKGROUND

### Tooth Structure

Human teeth serve several functions, including chewing, aiding in speech, and the perception of beauty and facial harmony. A human tooth consists of three sequential layers of tissues: (1) the hard, highly mineralized tissue, the “enamel”, supported by the less mineralized and vital connective tissue, (2) the “dentin”, which is formed from and supported by soft, connective tissue, and (3) the “dental pulp” or the “pulp”. The pulp consists of sensitive tissue containing blood vessels, nerve fibers, specialized cells and pulpal fluid. The dentin, which surrounds the dental pulp, forms the major part of the tooth. It is dense bonelike tissue consisting of 70% inorganic material, 20% organic material, and 10% water by weight.

### Dentin

Dentin is a calcified tissue of the body and, along with enamel, cementum, and pulp, is one of the four major components of teeth. It is usually covered by enamel on the crown and cementum on the root and surrounds the entire pulp. By volume, 45% of dentin consists of the mineral hydroxyapatite, 33% is organic material, and 22% is water. Yellow in appearance, it greatly affects the color of a tooth due to the translucency of enamel. Dentin, which is less mineralized and less brittle than enamel, is necessary for the support of enamel. There are two main characteristics which distinguish dentin from enamel: firstly, dentin forms throughout life; secondly, dentin is sensitive and can become hypersensitive to changes in temperature due to the sensory function of odontoblasts, especially when enamel recedes, and dentin channels become exposed.

Dentin may be demineralized and stained for histological study. Dentin consists of microscopic channels, called dentinal tubules, which radiate outward through the dentin from the pulp to the exterior cementum or enamel border. The dentinal tubules extend from the dentinoenamel junction (DEJ) in the crown area, or dentinocemental junction (DCJ) in the root area, to the outer wall of the pulp. From the outer surface of the dentin to the area nearest the pulp, these tubules follow an S-shaped path. Within the tubules, there is an odontoblast process, which is an extension of an odontoblast, and dentinal fluid, which contains a mixture of albumin, transferrin, tenascin and proteoglycans. In addition, there are branching canalicular systems that connect to each other. The major branches are the terminal ends of the tubules. The dentinal tubules contain the cytoplasmic extensions of odontoblasts that once formed the dentin and maintain it. Because of dentinal tubules, dentin has a degree of permeability, which can increase the sensation of pain and the rate of tooth decay.

Dentin is a bone-like matrix that is porous and yellow hued material. It is made up, by weight, of 70-72% inorganic materials (mainly hydroxylapatite and some non-crystalline amorphous calcium phosphate), 20% organic materials (90% of which is collagen type 1 and the remaining 10% ground substance, which includes dentin-specific proteins), and 8-10% water (which is adsorbed on the surface of the minerals or between the crystals). Because it is softer than enamel, it decays more rapidly and is subject to severe cavities if not properly treated, but due to its elastic properties, it is good support for enamel. Its flexibility prevents the brittle enamel fracturing.

### Remineralization and Demineralization of Hard Tissue

The process of the dissolution of enamel is called demineralization. It is the result of the interaction of the enamel and dentin components with the acid, produced by the bacterial action of plaque and various foods, as well as by the consumption of acidic beverages, such as fruit juices, wine and some sports and carbonated drinks. The decrease in pH results in the dissolution of Ca and P ions into the saliva. The solubility in acid of different types of the apatite found in the dentin and enamel varies significantly. For example, the solubility of carbonate apatite in an acid with a given pH is approximately an order of magnitude greater than that of hydroxyapatite, which, in turn, is an order of magnitude greater than that of fluorapatite. Demineralization leads to common dental disorders including development of caries, appearance of white spot lesions, and development of dentin hypersensitivity. Caries affect up to 90%+ of all adults in the United States. Caries are the result of bacterial acid-induced demineralization damage to tooth dentin. White spot lesions (WSL) affect approximately 50% of orthodontic patients. WSL result from localized demineralization caused by biofilm accumulation around orthodontic brackets. Dentin hypersensitivity (DH) affects approximately 11.5% of adults. DH is caused by the exposure of dentinal tubules (shown in FIG. 2), due to the loss of dentin and can also be caused by recession of the gum tissue around the base of a tooth.

The reverse process is called remineralization, which is facilitated by some or all of the following mechanisms. Human saliva contains calcium and phosphate in a supersaturated state, which can remineralize hydroxyapatite crystals lost during demineralization. This is the fundamental process in the prevention of dentin loss. Under normal conditions, there is a balance between demineralization and remineralization. The remineralizing ability of saliva is a typical example of the natural tooth rejuvenation mechanism. The remineralization process can also be initiated by controlling an oral fluid. The resistance of teeth to an acid attack can be increased and such methods as the use of fluoride in toothpastes and community water supplies have been known for many years. Fluorine ions from compounds, such as NaF and SnF₂, replace some of the OH-ions in apatite during the remineralization process. Amorphous calcium phosphate (CaPO₄) or ACP, is another compound used to promote remineralization. As the pH falls, ACP dissociates to form calcium and phosphate ions, thereby minimizing the drop in the pH and limiting demineralization. Since ACP can act as a reservoir for calcium and phosphate ions and maintain these ions in a state of supersaturation, ACP decreases the process of demineralization and promotes remineralization. Remineralized complexes consisting of Ca and F have been suggested as additives to strips and filling material.

Given the shortcomings of the traditional fluoride-based and calcium phosphate systems, methods and materials that aid regeneration or renewal and/or repair of lost or damaged hard tissue is needed.

### Acid Etching

Acid etching or tooth conditioning has a widespread use in clinical practice. It is most frequently used in bonding of resin materials. Different types and concentrations of acid may be used. Of these, 30-40% phosphoric acid with an application time of up to 60 seconds is the one most frequently used. Another, less frequent acid application is the removal of the superficial stains resulting from the developmental disturbances of the enamel or dentin, such as excessive intake of fluoride. Reported uses involve 18% and 37% hydrochloric acid applied for up to 25 seconds.

Acid etching and partial demineralization of apatite crystals leads to the high porosity of exposed surfaces, which makes such surfaces better suited for bonding of the restorative and adhesive materials. Three distinct acid etching patterns can be distinguished. A type I pattern is the one where the enamel rod cores are preferentially removed. In the type II pattern mostly prism sheaths are removed, while the rod cores remain intact. The type III pattern is characterized by irregular and indiscriminate etching.

Acid etching of hard tissue is a cause of the enamel or dentin loss and the decrease of mechanical hardness and wear resistance. In addition, acid etching of the superficial tooth layer, which is the most resistant to acid attack, can accelerate the growth of a carious lesion. For this reason, acid is used in dentistry mainly for the treatment of hard tissues to facilitate adhesion of tooth colored restorative materials to such hard tissues. In low concentrations, an acid is used as an addition to peroxide bleaching agents and some rinses and toothpastes for the stabilization of various ingredients. Dentists recommend limiting the use of acidic beverages and foods. Most foods and beverages have a pH of 2.5 or more, usually between 4 and 7.

### Tooth Rejuvenation

Tooth rejuvenation is one of the most important parts of preventive and esthetic dentistry. As explained above, it can be a part of the natural process, facilitated by the saliva. However, in many cases the natural role of the saliva may not be enough to keep a tooth from degradation. Several methods aimed to enhance tooth rejuvenation exist. Most are focused on the improvement of one the components of tooth rejuvenation, and do not provide a complete solution. Such methods are: water fluoridation, mouth rinses, gels and strips, tooth brushing, professional oral cleaning, tooth whitening, tooth coating, tooth surface laser modification. These methods are described below in more detail.

Water fluoridation contributes to the formation of fluorapatite in the external layer of the tooth. Fluoride in water plays several roles in the prevention of dental caries, such as the inhibition of acid production in plaque, the enhancement of remineralization of carious lesions and strengthening the tooth against an acid attack through the formation of the fluorapatite (Ca₁₀(PO₄)₆F₂). This effect takes place at low concentrations of fluoride. High concentrations of fluoride can cause the formation of CaF₂ and the destruction of tooth structure.

Mouth rinses are mainly used for bacterial reduction. Some additives, such as the casein phosphopeptide amorphous calcium phosphate nanocomplexes, have been proven to be effective in the remineralization process.

Different types of gels and strips and have been shown to provide an antibacterial effect. A gel, containing fluoride, calcium and phosphate ions, has been shown to be effective in the remineralization process. Preliminary treatment of the tooth with low acid concentrations enhances the effect of the fluoride treatment. Gels or strips may also include peroxide for tooth whitening.

Tooth brushing and flossing are the most important forms of preventing tooth stains and destruction of teeth since they are daily regimens. The mechanical cleaning of the teeth removes a biofilm, prevents/decreases the build-up of tartar and decreases acid production by bacteria. It also enhances the access of saliva to the tooth surface, in the process improving the chances for remineralization. In addition, toothpastes often contain antibacterial, remineralizing and whitening components.

Professional oral cleaning in the dental office provides additional benefits to the methods of tooth brushing and flossing, such as the removal of supra and subgingival plaque and calculus, plaque detection, and application of caries-preventing agents. The treatment typically involves the procedures, such as scaling and polishing of teeth and subgingival currettage, resulting in a more effective method of preventing of periodontal or other dental decreases, as well as an overall aesthetic improvement in the appearance of teeth and gums. Plaque detection and the application of the caries-preventing agents may also be performed by the health professional as an aid to home care and remineralization. However, this treatment is not capable of removing intrinsic and deep extrinsic stains.

Teeth function in an environment of mechanical, chemical and thermal stress. With normal chewing, a modest stress of 20 MPa is applied to the tooth more than 1000 times a day. Occasional stress can be up to 100 MPa. This cyclic loading occurs in a water-based fluid environment that can have a pH from 0.5 to 8 and the temperature variations of 50° C. Many different restorative materials have been developed, designed to retain their strength and properties in an aggressive environment (for example, ceramic-based porous alumina infiltrated with lanthanum aluminosilicate glass, or porous zirconia later infiltrated with glass). Porcelain, the most popular material, has excellent color properties, but is brittle and relatively easily fractured unless it is reinforced or strengthened. Porcelain restoration treatment also destroys the tooth structure since it usually requires tooth preparation and is expensive and time consuming. These restorative materials are used for crowns or veneers and, if done properly, provide excellent aesthetic appearance and prevent caries. However, the risk of recurrent caries still exists. Since any destruction of the tooth substance is harmful, clinicians have been attempting to develop non-destructive, or minimally destructive methods for tooth restoration.

### SUMMARY OF THE INVENTION

In certain aspects, the present disclose is directed to a method for remineralizing tooth dentin that involves contacting the tooth dentin with a catechol-containing material comprising one or more of catechol, semi-quinone, or quinone. The catechol-containing material can include monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of: a) a reactive species separates from the catechol or catechol containing material; and b) a catalyst, co-catalyst or an accelerator.

In certain aspects, the disclosure is directed to a method for treating dental hypersensitivity in a subject comprising contacting exposed dentin tubules with a catechol-containing material comprising catechol, semi-quinone, or quinone.

In certain aspects, the disclosure provides methods for restoring acid-etched tooth dentin in a subject comprising contacting the tooth dentin with a catechol-containing material.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 depicts a schematic showing the transition between demineralization and remineralization of a tooth in an acidic environment versus in normal saliva. This illustration (193069566° C.) was obtained with permission from Yomogil Dreamstime.com.

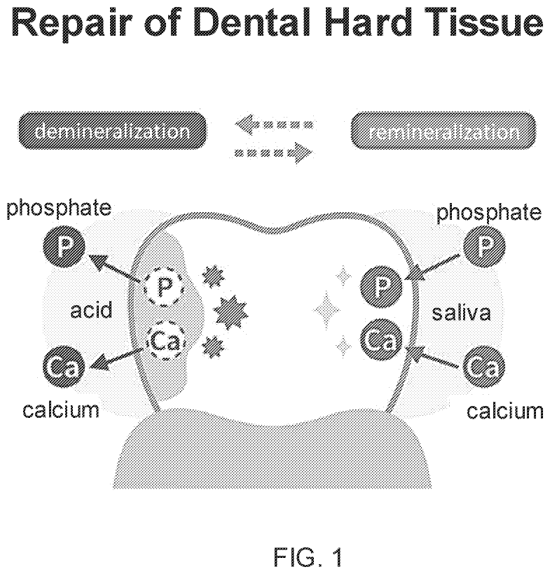

FIG. 2 depicts a schematic of a tooth with exposed dentin tubules which is a cause of dentin hypersensitivity.

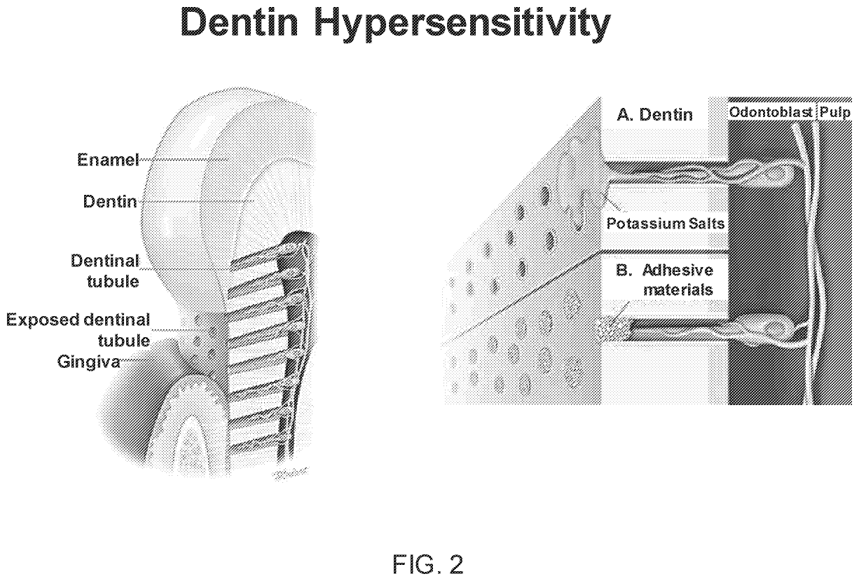

FIGS. 3A-3D depict Calcium Phosphate (CaP) Formation on PCS-Coated Aluminum. FIGS. 3A and 3B depict SEM micrographs of aluminum surface either (A, C) without treatment (FIG. 3A) or (B, D) with (FIG. 3B) a 10% PCS coating. FIGS. 3C and 3D depict EDX analysis of aluminum surface either (FIG. 3C) without treatment or with (FIG. 3D) a 10% PCS coating. The EDX analysis revealed this material to be dominantly CaP, with an average Ca/P molar ratio of 1.24±0.05 (n=10). Scale bars on SEM images are 10 microns.

FIGS. 4A-4F depict PCS-Induced Remineralization of Dentin. Samples of molars were ground to expose smooth dentin, demineralized with limited acid etching, coated with 5% Poly(Catechol-Styrene), and incubated in distilled water or 1.5× Simulated Body Fluid (SBF) for 72 hours at 37° C. The samples were then analyzed by SEM, and a 10-micron bar is shown for scale. FIG. 4A depicts distilled water controls showing normal dentin. FIG. 4B depicts 1.5×SBF control showing some CaP on the dentin surface. FIG. 4C depicts 5% PCS samples showing robust CaP formation inside tubules and in a sheet on dentin. FIGS. 4D, 4E, and 4F show energy dispersive X-Ray spectroscopy data confirming formation of ACP in the samples shown in FIGS. 4A, 4B, and 4C. The scale bar is 10 microns in FIGS. 4A, 4B, and 4C.

FIG. 5 is an SEM micrograph of dentin tubules treated with a thin film layer of 1% Poly(Catechol-Styrene) (PCS) showing mineralization of individual tubules. Nucleation of individual tubules can be seen. This nucleation growth is coming out of and going into the individual tubules eventually leading to total occlusion. Magnification is 12 kx and scale bar is 2.5 microns. The sample was acid etched with 40% phosphoric acid gel for two minutes, PCS was applied, and then submerged in 1.5× Simulated Body Fluid (SBF) at 37° C. for 72 hours.

FIGS. 6A and 6B are SEM micrographs of a dentin surface without treatment or treated with a thin film layer of 0.1% Poly(Catechol-Styrene) (PCS) showing 0.1% PCS is sufficient for mineralization. In FIG. 6A, the dentin was left untreated with PCS, and the micrographs shows very small patches of CaP growth. FIG. 6B is a closeup of the growth and shows light CaP and sporadically bound to the dentin surface. FIG. 6A has a magnification of 40× and scale bar is 1 millimeter, and FIG. 8B has a magnification of 800× and scale bar is 50 microns. Samples were acid etched with 40% phosphoric acid gel for two minutes, PCS was applied to the corresponding groups, and then samples were submerged in 1.5× Simulated Body Fluid (SBF) at 37° C. for 72 hours.

FIGS. 7A to 7D are SEM micrographs of a dentin surface without any treatment and treated with a thin film layer of 1% Poly(Catechol-Styrene) (PCS) showing growth patterns of amorphous calcium phosphate after re-acid etching. In FIGS. 7A and 7C, the samples were acid etched, submerged in 1.5×SBF for 72 hours, and then acid etched again. FIG. 7A shows and SEM of a sample where no PCS was applied. FIG. 7C shows a sample where a 1% PCS thin film was applied, and some residual CaP remains on the surface after re-acid etching (not shown); however, the tubules are exposed, and the acid etching has destroyed parts of the polymer coating. In FIGS. 7B and 7D, the dentin surface was acid etched, submerged in 1.5×SBF for 72 hours, acid etched again, and then resubmerged in 1.5×SBF for 72 hours. As shown in FIG. 7B, a CaP sheet growth was observed on the untreated surface. FIG. 7D shows that a larger amount of CaP sheet growth was observed on the 1% PCS treated surface even with parts of the PCS coating destroyed by the acid etching. All acid etchings were 2 min in length a with a 40% phosphoric acid gel. Scale bars of panels A, B, C, and D are 1 millimeter.

FIGS. 8A and 8B are SEM micrographs of a dentin surface without any treatment and treated with a thin film layer of 1% Poly(Catechol-Styrene) (PCS) depicting growth of amorphous calcium phosphate on a non-acid etched dentin surface. FIG. 8A shows an untreated dentin surface showing exposed tubules (inset). FIG. 8B shows the entire dentin surface covered in an amorphous calcium phosphate (ACP) sheet formation. Magnification is 80× and scale bars are 500 microns for each panel. FIGS. 8A and 8B have a magnified view of 80× magnification and scale bars are 500 microns. Samples were kept at 37° C. and submerged in 1.5× Simulated Body Fluid (SBF) for 72 hours.

FIGS. 9A to 9C are SEM micrographs of a dentin surface treated with a thin film layer of 5% Poly(Catechol-Styrene) (PCS) showing that the removal of the CaP sheet reveals occluded tubules underneath. The sample was incubated at 37° C. for 72 hours in 1.5× Simulated Body Fluid (SBF) and then placed into a cola bath for 60 seconds. FIG. 9A shows that the cola bath removed a section of the CaP sheet covering the dentin tubules. FIGS. 9B and 9C show occluded tubules that were observed in the area of the removed CaP sheet, with the 5% PCS layer still intact. FIG. 9A has a magnification of 80× and scale bar is 500 microns, FIG. 9B has a magnification of 800× and scale bar is 50 microns, FIG. 9C has a magnification of 2.5 kx and scale bar is 20 microns.

FIGS. 10A to 10D are SEM micrographs showing PCS-induced mineralization of dentin where mineralization appears enhanced in dentinal tubules. Samples of molars were ground to expose smooth dentin, demineralized with limited acid etching, coated with 5% PCS, and incubated in distilled water or 1.5× Simulated Body Fluid (SBF) for 72 hours/37° C. The samples were then analyzed by SEM, and a 10-micron bar is shown for scale. Representative images were taken from areas of low (FIG. 10A), intermediate (FIGS. 10B and 10C, and high remineralization (FIG. 10D). Results suggest that remineralization appears to start within the dentinal tubules.

### DETAILED DESCRIPTION OF ILLUSTRATIVE EMBODIMENTS

The present disclosure provides methods for tooth rejuvenation including remineralization of tooth structures such as tooth dentin. The methods also provide tooth protection from environmental conditions that may cause damage or demineralization of tooth dentin. The methods provided herein also provide for improvement of the mechanical and chemical resistance of tooth substance and to improve its aesthetic appearance. Tooth rejuvenation is defined as the changing of the tooth structure leading to an increase in some or all of the following parameters: wear resistance (mechanical resistance), resistance to chemical and/or bacterial attack, and the restoration and improvement of tooth appearance and other tooth improvements. One of the embodiments of the present disclosure is a method for tooth rejuvenation comprising applying a layer catechol styrene-based composition to a tooth. In one embodiment, the tooth dentin can be mineralized using catechol containing compound such as poly(catechol-styrene).

### Definitions

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

In the present disclosure, the term “subject” includes any human or non-human animal. In certain embodiments, the subject is a human or non-human mammal. In certain embodiments, the subject is a human.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one nonlimiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For ex-ample, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosure which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

Catechol is able to form a wide range of reversible bonds with surfaces, such as hydrogen bonding, cation-π interaction, and metal ion complexation. Catechol can also form covalent bonds with certain bonding surfaces, for example protein surfaces, as well as cross-linking with itself.

Catechol groups are susceptible to oxidation into a semi-quinone or quinone. Several studies have documented that oxidation reduces significantly binding to inorganic surfaces. During the process of catechol oxidation into quinones, reactive oxygen species (ROS) are generated as by-products.

While strong attachment to inorganic surfaces requires catechol (reduced), quinones (oxidized) can bind covalently to organic materials via a Schiff base addition or Michael Reaction. Mussel-inspired biomimetic adhesives can be considered bifunctional “catechol-quinone” mixtures.

The catechol containing materials of the present invention are used for remineralization of dentin. Catechol containing materials of the present invention are also used for remineralization of dentin.

In an aspect, the disclosure is directed to a process of remineralization of the dentin wherein the catechol containing material comprises a catechol containing monomer, polymer, or oligomer, wherein said catechol presents as a catechol and/or as a semi-quinone and/or as a quinone with or without the presence of a primary amine or a secondary amine; and wherein the polymeric layer also comprises a reactive material that is not reactive with catechol or quinone.

In an aspect, the present invention is directed to a polymeric layer, comprising a catechol containing monomer, polymer, or oligomer, wherein said catechol presents as a catechol and/or as a semi-quinone and/or as a quinone without the presence of a primary amine or a secondary amine; and wherein the polymeric layer optionally comprises a reactive material that is not reactive with catechol or quinone.

In some embodiments, the catechol containing monomer, polymer, or oligomer in the polymeric layer is monomeric. In some embodiments, the catechol containing monomer, polymer, or oligomer in the polymeric layer is oligomeric. In some embodiments, the catechol containing monomer, polymer, or oligomer in the polymeric layer is polymeric.

In some embodiments, the catechol-containing material comprises the reactive species separate from the catechol or catechol containing material; and the reactive species is an acrylic such as 2,2-bis[4-(2-hydroxy-3-methacryloyloxypropoxy)phenyl] propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic di-methacrylate, oxydiphthalic acid dimethacrylate, phenyl dihydroxymethacrylate diphosphonate, Acidic Bisphenol-A dimethacrylate, morpholine carbonyl methacrylate, phenyl carbonate methacrylate.

In some embodiments, the catechol-containing material includes free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

In some embodiments, the acrylate is an acrylate monomer comprising a vinyl group and at least one of a carboxylic acid ester and a carboxylic acid nitrile; and wherein the acrylate is linear or branched. In some embodiments, the acrylate is ethyl acrylate, ethylene-methyl acrylate, methyl methacrylate, 2-chloroethyl vinyl ether, 2-hydroxyethyl acrylate, hydroxyethyl methacrylate, butyl acrylate, trimethylolpropane triacrylate (TMPTA) or combinations thereof.

In an aspect, the catechol-containing material has a thickness of from about 10 nanometers to about 100 microns. In some embodiments, the catechol-containing material has a thickness of from about 15 nanometers to about 50 microns. In some embodiments, the remineralization polymeric layer has a thickness of from about 15 nanometers to about 15 microns. In some embodiments, the remineralization polymeric layer has a thickness of from about 150 nanometers to less than about 15 microns. In some embodiments, the remineralization polymeric layer has a thickness of from about 150 nanometers to about 1.5 microns.

In some embodiments, the remineralization polymeric layer has a thickness of from about 10 nanometers to about 100 microns; or from about 10 nanometers to about 100 nanometers; or from about 100 nanometers to about 150 nanometers; or from about 150 nanometers to about 200 nanometers; or from about 200 nanometers to about 250 nanometers; or from about 250 nanometers to about 300 nanometers; or from about 300 nanometers to about 350 nanometers; or from about 350 nanometers to about 400 nanometers; or from about 400 nanometers to about 450 nanometers; or from about 450 nanometers to about 500 nanometers; or from about 500 nanometers to about 550 nanometers; or from about 550 nanometers to about 600 nanometers; or from about 600 nanometers to about 650 nanometers; or from about 650 nanometers to about 700 nanometers; or from about 700 nanometers to about 750 nanometers; or from about 750 nanometers to about 800 nanometers; or from about 800 nanometers to about 850 nanometers; or from about 850 nanometers to about 900 nanometers; or from about 900 nanometers to about 950 nanometers; or from about 950 nanometers to about 1000 nanometers.

In some embodiments, the remineralization polymeric layer has a thickness of from about 1 micron to about 1.5 microns; or from about 1.5 microns to about 5 microns; or from about 5 microns to about 10 microns; or from about 10 microns to about 15 microns; or from about 15 microns to about 20 microns; or from about 20 microns to about 25 microns; or from about 25 microns to about 30 microns; or from about 30 microns to about 35 microns; or from about 35 microns to about 40 microns; or from about 40 microns to about 45 microns; or from about 45 microns to about 50 microns; or from about 50 microns to about 55 microns; or from about 55 microns to about 60 microns; or from about 60 microns to about 65 microns; or from about 65 microns to about 70 microns; or from about 70 microns to about 75 microns; or from about 75 microns to about 80 microns; or from about 80 microns to about 85 microns; or from about 85 microns to about 90 microns; or from about 90 microns to about 95 microns; or from about 95 microns to about 100 microns.

In an aspect, the catechol containing monomer, polymer, or oligomer in the remineralization polymeric layer comprises poly-catechol styrene (PCS).

In some embodiments, the PCS is prepared in one or more suitable solvents. For example, the PCS may be prepared as a solution in acetone, tert-butyl alcohol, ethanol, isopropyl alcohol, or a combination thereof, or one or more other suitable solvents as understood in the art. In some embodiments, the PCS is prepared as a solution in acetone. In some embodiments, the PCS is prepared as a solution in tert-butyl alcohol. In some embodiments, the PCS is prepared as a solution in isopropyl alcohol. In some embodiments the PCS is prepared as a solution in ethanol.

In some embodiments, the PCS comprises a solution containing from about 0.001% to 10% PCS, from about 0.05% to about 5% PCS, from about 0.01% to about 2% PCS, from about 0.5% to about 1% PCS, from about 0.1% to about 0.5% PCS and any and all increments therebetween. In some embodiments, the PCS comprises about 0.1% catechol.

In some embodiments, the PCS comprises from about 20% catechol to about 22% catechol; or from about 22% catechol to about 24% catechol; or from about 24% catechol to about 26% catechol; or from about 26% catechol to about 28% catechol; or from about 28% catechol to about 30% catechol; or from about 30% catechol to about 32% catechol; or from about 32% catechol to about 34% catechol; or from about 34% catechol to about 36% catechol; or from about 36% catechol to about 38% catechol; or from about 38% catechol to about 40% catechol.

In some embodiments, the remineralization polymeric layer comprises a reactive material that is not reactive with catechol or quinone. In some embodiments, the reactive material is not reactive at ambient temperature with catechol or quinone. In some embodiments, the reactive material is not reactive at low temperature with catechol or quinone.

In some embodiments, the reactive material that is not reactive with catechol or quinone is a resin, an oligomer, a polymer, or a monomer. In some embodiments, the reactive material an oligomer. In some embodiments, the reactive material a polymer. In some embodiments, the reactive material a monomer.

In an aspect, the remineralization polymeric layer is a continuous layer. In an aspect, the remineralization polymeric layer is a non-continuous layer. In an aspect, the remineralization polymeric lay-er is a patterned layer or a textured layer.

In some embodiments, the remineralization polymeric layer includes one or more additives. In some embodiments, the one or more additives include one or more catalysts, for example one or more photo-initiators. The one or more photo-initiators may include one or more of camphorquinone (CQ), azobisisobutyronitrile (AIBN), benzoyl peroxide, 2,2-dimethoxy-2-phenylacetophenone, and one or more combinations thereof. In some embodiments, the photo-initiator may include one or more additional photosensitizers or co-initiators including for example one or more peroxides, aliphatic azo compounds and the like. In some embodiments, the catalyst, co-catalyst or accelerator; and the catalyst, co-catalyst or accelerator is an acrylate catalyst that promotes an acrylate polymerization reaction, or combinations thereof.

In some embodiments, the photo-initiator is CQ. The CQ may be used at a concentration of from about 0.01% to about 1%, from about 0.05% to about 0.75% from about 0.1% to about 0.5%, and any and all increments therebetween. In some embodiments the CQ is used at a concentration of 0.1%.

In some embodiments, the catechol containing monomer, oligomer, or polymer comprises PCS.

In some embodiments, the polymeric layer on the dentin is applied as a solution. In some embodiments, the solution comprises from about 0.001% by weight to about 10% by weight of the catechol containing monomer, polymer, or oligomer. In some embodiments, the solution comprises from about 0.01% by weight to about 5% by weight of the catechol containing monomer, polymer, or oligomer. In some embodiments, the solution comprises from about 0.01% by weight to about 1% by weight of the catechol containing monomer, polymer, or oligomer. In some embodiments, the solution comprises from about 0.1% by weight to about 1% by weight of the catechol containing monomer, polymer, or oligomer.

In some embodiments, the solution comprises from about 0.001% by weight to about 0.005% by weight of the catechol containing monomer, polymer, or oligomer; or from about 0.005% by weight to about 0.01% by weight of the catechol containing monomer, polymer, or oligomer; or from about 0.01% by weight to about 0.02% by weight of the catechol containing monomer, polymer, or oligomer; or from about 0.02% by weight to about 0.03% by weight of the catechol containing monomer, polymer, or oligomer; or from about 0.03% by weight to about 0.04% by weight of the catechol containing monomer, polymer, or oligomer; or from about 0.04% by weight to about 0.05% by weight of the catechol containing monomer, polymer, or oligomer; or from about 0.05% by weight to about 0.06% by weight of the catechol containing monomer, polymer, or oligomer; or from about 0.06% by weight to about 0.07% by weight of the catechol containing monomer, polymer, or oligomer; or from about 0.07% by weight to about 0.08% by weight of the catechol containing monomer, polymer, or oligomer; or from about 0.08% by weight to about 0.09% by weight of the catechol containing monomer, polymer, or oligomer; or from about 0.09% by weight to about 0.1% by weight of the catechol containing monomer, polymer, or oligomer; or from about 0.1% by weight to about 0.11% by weight of the catechol containing monomer, polymer, or oligomer; or from about 0.11% by weight to about 0.12% by weight of the catechol containing monomer, polymer, or oligomer; or from about 0.12% by weight to about 0.13% by weight of the catechol containing monomer, polymer, or oligomer; or from about 0.13% by weight to about 0.14% by weight of the catechol containing monomer, polymer, or oligomer; or from about 0.14% by weight to about 0.15% by weight of the catechol containing monomer, polymer, or oligomer; or from about 0.15% by weight to about 0.2% by weight of the catechol containing monomer, polymer, or oligomer; or from about 0.2% by weight to about 0.25% by weight of the catechol containing monomer, polymer, or oligomer; or from about 0.25% by weight to about 0.3% by weight of the catechol containing monomer, polymer, or oligomer; or from about 0.3% by weight to about 0.35% by weight of the catechol containing monomer, polymer, or oligomer; or from about 0.35% by weight to about 0.4% by weight of the catechol containing monomer, polymer, or oligomer; or from about 0.4% by weight to about 0.45% by weight of the catechol containing monomer, polymer, or oligomer; or from about 0.45% by weight to about 0.5% by weight of the catechol containing monomer, polymer, or oligomer; or from about 0.5% by weight to about 0.75% by weight of the catechol containing monomer, polymer, or oligomer; or from about 0.75% by weight to about 1% by weight of the catechol containing monomer, polymer, or oligomer; or from about 1.25% by weight to about 1.5% by weight of the catechol containing monomer, polymer, or oligomer; or from about 1.5% by weight to about 1.75% by weight of the catechol containing monomer, polymer, or oligomer; or from about 1.75% by weight to about 2% by weight of the catechol containing monomer, polymer, or oligomer.

In some embodiments, the catechol containing monomer, polymer, or oligomer used in the solution is poly-catechol styrene (PCS). In some embodiments, the solution comprises from about 0.001% by weight to about 10% by weight of PCS. In some embodiments, the solution comprises from about 0.01% by weight to about 5% by weight of PCS. In some embodiments, the solution comprises from about 0.01% by weight to about 1% by weight of PCS. In some embodiments, the solution comprises from about 0.1% by weight to about 1% by weight of PCS.

In some embodiments, the solution comprises from about 0.001% by weight to about 0.005% by weight of PCS; or from about 0.005% by weight to about 0.01% by weight of PCS; or from about 0.01% by weight to about 0.02% by weight of PCS; or from about 0.02% by weight to about 0.03% by weight of PCS; or from about 0.03% by weight to about 0.04% by weight of PCS; or from about 0.04% by weight to about 0.05% by weight of PCS; or from about 0.05% by weight to about 0.06% by weight of PCS; or from about 0.06% by weight to about 0.07% by weight of PCS; or from about 0.07% by weight to about 0.08% by weight of PCS; or from about 0.08% by weight to about 0.09% by weight of PCS; or from about 0.09% by weight to about 0.1% by weight of PCS; or from about 0.1% by weight to about 0.11% by weight of PCS; or from about 0.11% by weight to about 0.12% by weight of PCS; or from about 0.12% by weight to about 0.13% by weight of PCS; or from about 0.13% by weight to about 0.14% by weight of PCS; or from about 0.14% by weight to about 0.15% by weight of PCS; or from about 0.15% by weight to about 0.2% by weight of PCS; or from about 0.2% by weight to about 0.25% by weight of PCS; or from about 0.25% by weight to about 0.3% by weight of PCS; or from about 0.3% by weight to about 0.35% by weight of PCS; or from about 0.35% by weight to about 0.4% by weight of PCS; or from about 0.4% by weight to about 0.45% by weight of PCS; or from about 0.45% by weight to about 0.5% by weight of PCS; or from about 0.5% by weight to about 0.75% by weight of PCS; or from about 0.75% by weight to about 1% by weight of PCS; or from about 1.25% by weight to about 1.5% by weight of PCS; or from about 1.5% by weight to about 1.75% by weight of PCS; or from about 1.75% by weight to about 2% by weight of PCS.

In some embodiments, the solution also comprises an aqueous or organic solvent for dissolving the catechol containing monomer, polymer, or oligomer. In some embodiments, the organic solvent is acetone, tert-butyl alcohol, ethanol, isopropyl alcohol, or a combination thereof.

In some embodiments, the organic solvent is acetone. In some embodiments, the organic solvent is tert-butyl alcohol. In some embodiments, the organic solvent is ethanol. In some embodiments, the organic solvent is isopropyl alcohol. In some embodiments, the organic solvent is a combination of one or more of acetone tert-butyl alcohol, ethanol, isopropyl alcohol. In some embodiments, the organic solvent is acetone and the catechol or catechol containing material is PCS. In some embodiments, the organic solvent is tert-butyl alcohol and the catechol or catechol containing material is PCS. In some embodiments, the organic solvent is ethanol and the catechol or catechol containing material is PCS. In some embodiments, the organic solvent is isopropyl alcohol and the catechol or catechol containing material is PCS. In some embodiments, the organic solvent is a combination of one or more of acetone tert-butyl alcohol, ethanol, isopropyl alcohol, and the catechol or catechol containing material is PCS. In some embodiments, the organic solvent further comprises one or more oxidizing agents or acidifying agents. For example, in some embodiments the organic solvent further comprises acetic acid.

The pH of the solution is not particularly limited. In some embodiments, the pH of the solution is about 3; or about 3.5; or about 4; or about 4.5; or about 5; or about 5.5; or about 6; or about 6.5; or about 7; or about 7.5; or about 8; or about 8.5; or about 9; or about 9.5; or about 10; or about 10.5; or about 11.

In some embodiments, the pH of the solution is from about 3 to 3.5; or about 3.5 to 4; or about 4 to 4.5; or about 4.5 to 5; or about 5 to 5.5; or about 5.5 to 6; or about 6 to 6.5; or about 6.5 to 7; or about 7 to 7.5; or about 7.5 to 8; or about 8 to 8.5; or about 8.5 to 9; or about 9 to 9.5; or about 9.5 to 10; or about 10 to 10.5; or about 10.5 to 11.

### Methods

In certain aspects, the present disclosure provides methods for remineralizing tooth dentin by contacting one or more surfaces of the tooth with one or more catechol-containing materials as contemplated herein. The one or more surfaces of the tooth can include the tooth dentin. The catechol-containing can include one or more of catechol, semi-quinone, or quinone. In some embodiments, the catechol or catechol containing material is PCS. The PCS can be applied in a solution comprising from about 0.001% by weight to about 10% by weight of PCS. In some embodiments, the solution comprises from about 0.01% by weight to about 5% by weight of PCS. In some embodiments, the solution comprises from about 0.01% by weight to about 1% by weight of PCS. In some embodiments, the solution comprises from about 0.1% by weight to about 1% by weight of PCS. In some embodiments, the solution comprises from about 0.001% by weight to about 0.005% by weight of PCS; or from about 0.005% by weight to about 0.01% by weight of PCS; or from about 0.01% by weight to about 0.02% by weight of PCS; or from about 0.02% by weight to about 0.03% by weight of PCS; or from about 0.03% by weight to about 0.04% by weight of PCS; or from about 0.04% by weight to about 0.05% by weight of PCS; or from about 0.05% by weight to about 0.06% by weight of PCS; or from about 0.06% by weight to about 0.07% by weight of PCS; or from about 0.07% by weight to about 0.08% by weight of PCS; or from about 0.08% by weight to about 0.09% by weight of PCS; or from about 0.09% by weight to about 0.1% by weight of PCS; or from about 0.1% by weight to about 0.11% by weight of PCS; or from about 0.11% by weight to about 0.12% by weight of PCS; or from about 0.12% by weight to about 0.13% by weight of PCS; or from about 0.13% by weight to about 0.14% by weight of PCS; or from about 0.14% by weight to about 0.15% by weight of PCS; or from about 0.15% by weight to about 0.2% by weight of PCS; or from about 0.2% by weight to about 0.25% by weight of PCS; or from about 0.25% by weight to about 0.3% by weight of PCS; or from about 0.3% by weight to about 0.35% by weight of PCS; or from about 0.35% by weight to about 0.4% by weight of PCS; or from about 0.4% by weight to about 0.45% by weight of PCS; or from about 0.45% by weight to about 0.5% by weight of PCS; or from about 0.5% by weight to about 0.75% by weight of PCS; or from about 0.75% by weight to about 1% by weight of PCS; or from about 1.25% by weight to about 1.5% by weight of PCS; or from about 1.5% by weight to about 1.75% by weight of PCS; or from about 1.75% by weight to about 2% by weight of PCS, including any and all increments therebetween. The PCS can be applied in a solution comprising from about 1% by weight to about 3% by weight of PCS, from about 2% by weight to about 5% by weight of PCS, from about 3% by weight to about 7% by weight of PCS, from about 4% by weight to about 10% by weight of PCS, and any and all increments therebetween.

The tooth surface may include a tooth surface that has undergone acid etching. The tooth surface may include a tooth surface that has not undergone acid etching. The acid etching can include acid etching using an acid such as phosphoric acid. The acid etching can include acid etching using an acid that is weaker than phosphoric acid including for example maleic acid, polyacrylic acid, or the like.

In certain aspects, the present disclosure provides method for treating dental hypersensitivity in a subject comprising contacting exposed dentin tubules with a catechol-containing material comprising catechol, semi-quinone, or quinone. The catechol-containing material can include PCS. The PCS may be applied in a solution comprising from about 0.001% by weight to about 10% by weight of PCS. In some embodiments, the solution comprises from about 0.01% by weight to about 5% by weight of PCS. In some embodiments, the solution comprises from about 0.01% by weight to about 1% by weight of PCS. In some embodiments, the solution comprises from about 0.1% by weight to about 1% by weight of PCS. In some embodiments, the solution comprises from about 0.001% by weight to about 0.005% by weight of PCS; or from about 0.005% by weight to about 0.01% by weight of PCS; or from about 0.01% by weight to about 0.02% by weight of PCS; or from about 0.02% by weight to about 0.03% by weight of PCS; or from about 0.03% by weight to about 0.04% by weight of PCS; or from about 0.04% by weight to about 0.05% by weight of PCS; or from about 0.05% by weight to about 0.06% by weight of PCS; or from about 0.06% by weight to about 0.07% by weight of PCS; or from about 0.07% by weight to about 0.08% by weight of PCS; or from about 0.08% by weight to about 0.09% by weight of PCS; or from about 0.09% by weight to about 0.1% by weight of PCS; or from about 0.1% by weight to about 0.11% by weight of PCS; or from about 0.11% by weight to about 0.12% by weight of PCS; or from about 0.12% by weight to about 0.13% by weight of PCS; or from about 0.13% by weight to about 0.14% by weight of PCS; or from about 0.14% by weight to about 0.15% by weight of PCS; or from about 0.15% by weight to about 0.2% by weight of PCS; or from about 0.2% by weight to about 0.25% by weight of PCS; or from about 0.25% by weight to about 0.3% by weight of PCS; or from about 0.3% by weight to about 0.35% by weight of PCS; or from about 0.35% by weight to about 0.4% by weight of PCS; or from about 0.4% by weight to about 0.45% by weight of PCS; or from about 0.45% by weight to about 0.5% by weight of PCS; or from about 0.5% by weight to about 0.75% by weight of PCS; or from about 0.75% by weight to about 1% by weight of PCS; or from about 1.25% by weight to about 1.5% by weight of PCS; or from about 1.5% by weight to about 1.75% by weight of PCS; or from about 1.75% by weight to about 2% by weight of PCS.

### ASPECTS

The following Aspects are illustrative only and do not limit the scope of the present disclosure or the appended claims. Any part or parts of any one or more Aspects can be combined with any part or parts of any one or more other Aspects.

Aspect 1: A method for remineralizing tooth dentin comprising contacting the tooth dentin with a catechol-containing material comprising one or more of catechol, semi-quinone, or quinone.

Aspect 2: The method of Aspect 1, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of: a) a reactive species separate from the catechol or catechol containing material; and b) a catalyst, co-catalyst or an accelerator.

Aspect 3: The method of Aspect 2, wherein the catechol-containing material comprises the reactive species separate from the catechol or catechol containing material; and the reactive species comprises an acrylic, a silane, a silicone, a methacrylate, a polyvinyl alcohol (PVA), a urethane, or a combination thereof.

Aspect 4: The method of Aspect 3, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

Aspect 5: The method of Aspect 1 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

Aspect 6: The method of Aspect 3, wherein the reactive species is the acrylate.

Aspect 7: The method of Aspect 1, wherein the catechol-containing material is disposed upon a tooth dentin.

Aspect 8: The method of Aspect 1, wherein the catechol-containing has a thickness of from about 10 nanometers to about 500 microns.

Aspect 9: The method of Aspect 1, wherein the catechol-containing material comprises about 0.1% poly(catechol-styrene) (PCS).

Aspect 10: The method of Aspect 1, wherein the catechol-containing material comprises an amount of PCS in the range of from about 0.1% to about 10%.

Aspect 11: The method of Aspect 1, wherein the catechol-containing material comprises at least partially oxidized PCS.

Aspect 12: The method of Aspect 1, wherein the catechol-containing material comprises at least partially reduced PCS.

Aspect 13: The method of Aspect 1, wherein the catechol-containing material comprises material having at least one partially intermediate oxidation state.

Aspect 14: A method for treating dental hypersensitivity in a subject comprising contacting exposed dentin tubules with a catechol-containing material comprising catechol, semi-quinone, or quinone.

Aspect 15: The method of Aspect 14, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of: a) a reactive species separate from the catechol or catechol containing material; and b) a catalyst, co-catalyst or an accelerator.

Aspect 16: The method of Aspect 15, wherein the catechol-containing material comprises the reactive species separate from the catechol or catechol containing material; and the reactive species comprises an acrylic, a silane, a silicone, a methacrylate, a polyvinyl alcohol (PVA) or a combination thereof.

Aspect 17: The method of Aspect 16, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

Aspect 18: The method of Aspect 14 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

Aspect 19: The method of Aspect 16, wherein the reactive species is the acrylate.

Aspect 20: The method of Aspect 14, wherein the catechol-containing material is disposed upon a tooth dentin.

Aspect 21: The method of Aspect 14, wherein the catechol-containing has a thickness of from about 10 nanometers to about 500 microns.

Aspect 22: The method of Aspect 14, wherein the catechol-containing material comprises about 0.1% poly(catechol-styrene) (PCS).

Aspect 23: The method of Aspect 14, wherein the catechol-containing material comprises an amount of PCS in the range of from about 0.1% to about 10%.

Aspect 24: The method of Aspect 14, wherein the catechol-containing material comprises at least partially oxidized PCS.

Aspect 25: The method of Aspect 14, wherein the catechol-containing material comprises at least partially reduced PCS.

Aspect 26: The method of Aspect 14, wherein the catechol-containing material comprises material having at least one partially intermediate oxidation state.

Aspect 27: A method for restoring acid-etched tooth dentin in a subject comprising contacting the tooth dentin with a catechol-containing material comprising catechol, semi-quinone, or quinone.

Aspect 28: The method of Aspect 27, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of: a) a reactive species separate from the catechol or catechol containing material; and b) a catalyst, co-catalyst or an accelerator.

Aspect 29: The method of Aspect 28, wherein the catechol-containing material comprises the reactive species separate from the catechol or catechol containing material; and the reactive species comprises an acrylic, a silane, a silicone, a methacrylate, a polyvinyl alcohol (PVA) or a combination thereof.

Aspect 30: The method of Aspect 29, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

Aspect 31: The method of Aspect 27 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

Aspect 32: The method of Aspect 29, wherein the reactive species is the acrylate.

Aspect 33: The method of Aspect 27, wherein the catechol-containing material is disposed upon a tooth dentin.

Aspect 34: The method of Aspect 27, wherein the catechol-containing has a thickness of from about 10 nanometers to about 500 microns.

Aspect 35: The method of Aspect 27, wherein the catechol-containing material comprises about 0.1% poly(catechol-styrene) (PCS).

Aspect 36: The method of Aspect 27, wherein the catechol-containing material comprises an amount of PCS in the range of from about 0.1% to about 10%.

Aspect 37: The method of Aspect 27, wherein the catechol-containing material comprises at least partially oxidized PCS.

Aspect 38: The method of Aspect 27, wherein the catechol-containing material comprises at least partially reduced PCS.

Aspect 39: The method of Aspect 27, wherein the catechol-containing material comprises material having at least one partially intermediate oxidation state.

### EXAMPLES

### Example 1: PCS-Induced Mineralization on Aluminum

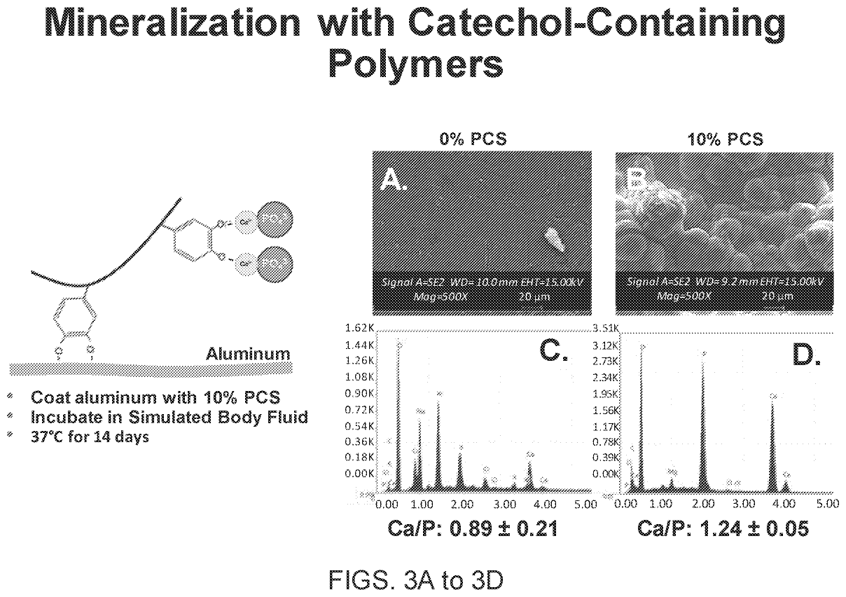

In order to determine if Poly(Catechol-Styrene) facilitates the formation of CaP in physiological conditions, a simple aluminum substrate was used. The aluminum was cleaned, coated with a thin layer of 10% PCS (w/v), immersed in 1.5× simulated body fluid (SBF), and incubated at 37° C. for 14 days. A white crystalline material was visible on the aluminum within 24 hours (data not shown). Examination with Scanning Electron Microscopy (SEM) and Energy-Dispersive X-ray Spectroscopy (EDS) (FIGS. 3A-3D) revealed that the induced layer was Calcium Phosphate (CaP).

### Example 2: PCS-Induced Mineralization of Dentin

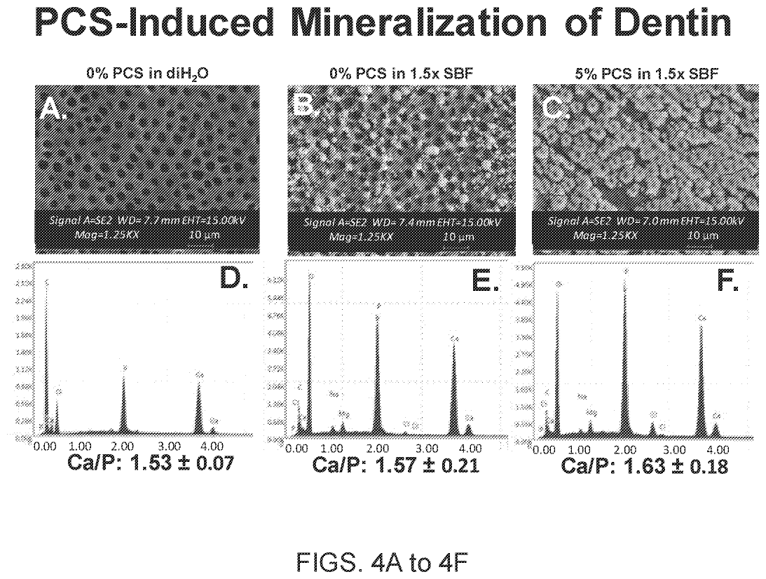

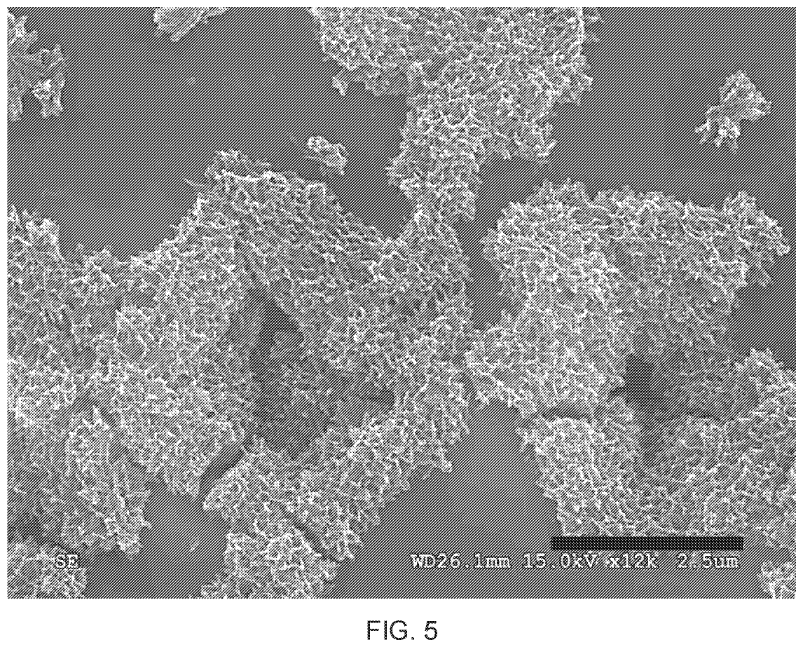

In order to determine if PCS could induce CaP formation on demineralized dentin, extracted molars were obtained from dental clinics, cleaned, and stored in chloramine T at 4° C. Teeth were mounted in resin and ground with 600-grit silicon carbide papers to obtain smooth dentin. They were then acid-etched with 40% phosphoric acid gel for 2 minutes, rinsed in diH2O, coated with PCS (5% in acetone), and incubated in 1.5×SBF for 3 days at 37° C. The samples were dried, sputter coated, and analyzed with SEM and EDS (FIGS. 4A-4F). Results indicate that PCS-induced formation of a CaP sheet covering exposed dentin. This sheet had a Ca/P of 1.63, close to the expected value for hydroxyapatite (HA). High magnification SEM revealed a crystalline form (FIG. 5). Without being bound to theory, it is possible that endogenous HA may have acted as a mineralization template. It was observed that dentinal tubules contain nucleation sites from which CaP grows outward in a uniform layer and coalesces into a sheet. Interestingly, striations (linear patterns in FIG. 4C) were observed. PCS may selectively adhere to proteins distributed in this striated pattern. These proteins may be rich in Cys (free-SH) and/or Lys (free-NH2), in which case oxidized PCS would bond covalently.

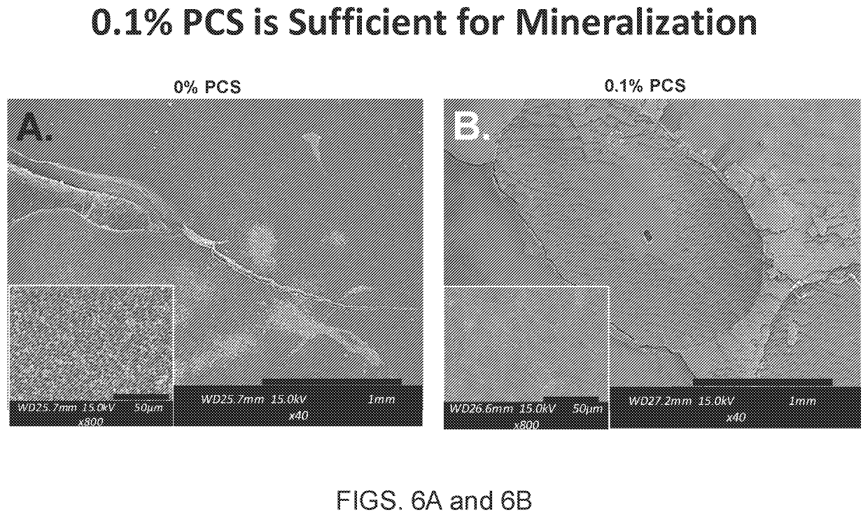

While initial studies were conducted with 5% or 10% PCS in acetone. It was set out to determine if mineralization could be achieved with less PCS. Accordingly, similar experiments were performed using 0.1% PCS in acetone. Results, shown in FIGS. 6A and 6B, indicate that when treated with a 0.1% PCS thin film, a large sheet of CaP growth can be seen on the dentin surface. The CaP is fused together to form a large sheet. These micrographs show that even 0.1% PCS is enough to produce a large, mineralized CaP sheet on the dentin surface.

### Example 3: PSC Resistance to Chemical and Physical Challenges

Remineralization after Re-Acid Etching

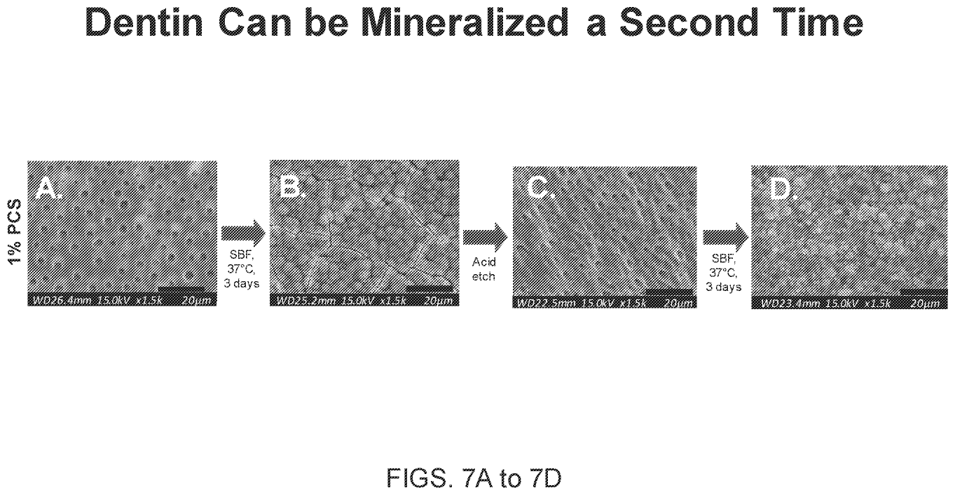

In order to determine whether PCS provides a protective effect upon chemical challenge, growth patterns of Calcium Phosphate (CaP) were evaluated after a second acid etching of dentin samples. Briefly, the samples were acid etched, a 1% PCS thin film was applied, and submerged in 1.5×SBF for 72 hours. As shown in FIG. 7B, CaP sheet growth was observed. Then samples were acid etched again. Some residual CaP remains on the surface after re-acid etching (FIG. 7C). However, the tubules were exposed, and the acid etching had destroyed parts of the polymer coating. The samples were then resubmerged in 1.5×SBF for 72 hours. As shown in FIG. 7D, a larger amount of CaP sheet growth was observed on the 1% PCS treated surface even with parts of the PCS coating destroyed by the acid etching. All acid etchings were 2 minutes in length a with a 40% phosphoric acid gel.

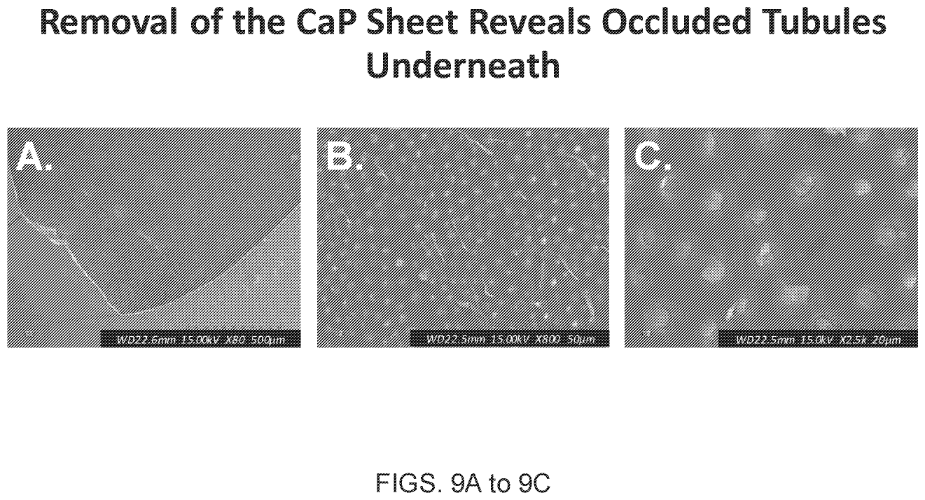

Furthermore, even though portions of the CaP sheet were destroyed by the acid treatment, results shown in FIGS. 9A-9C verify that dentin tubules are in fact occluded by the CaP sheet. This result provides strong evidence for use of PCS for use in treatment of dentin hypersensitivity, which results due to exposure of dentin tubules.

Remineralization without Acid Etching

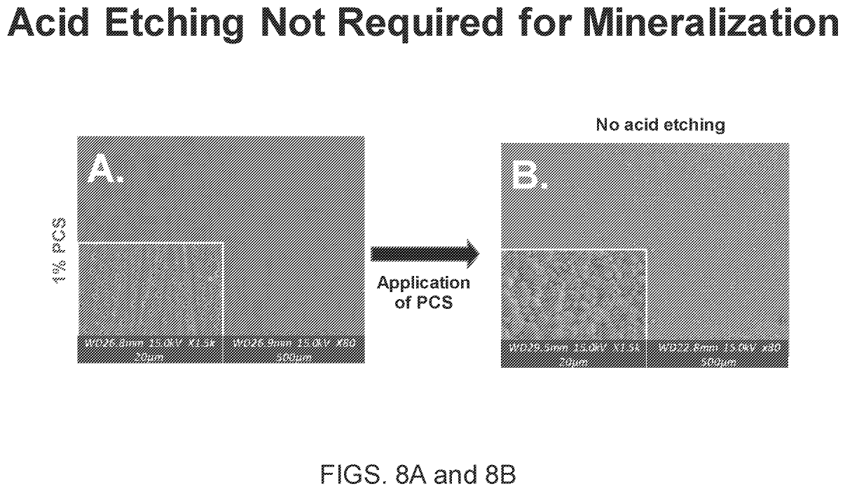

It is standard practice to perform acid washing or acid etching of teeth prior to dental procedures in order to prepare the dental surface and/or to clean the surface. In many cases an acid wash or acid preparatory step is required for certain detail adhesives or dental treatments to properly interact with the surface of teeth. Phosphoric acid is often used as a first pass acid. However, patients with tooth sensitivity may not tolerate an acid as strong as phosphoric acid, in which case a weaker acid such as maleic acid or polyacrylic acid may be used. The above experiments were performed following acid etching with strong acid as is standard practice. However, in order to determine whether remineralization with PCS requires acid etching, formation of CaP was evaluated in the absence of acid etching. Accordingly, SEM images of the dentin surface were acquired without acid etching. Results, shown in FIGS. 8A and 8B, indicated that the entire dentin surface is covered in a CaP sheet without requiring acid etching.

### Example 4: Patterns of Remineralization

### Localization of Remineralization

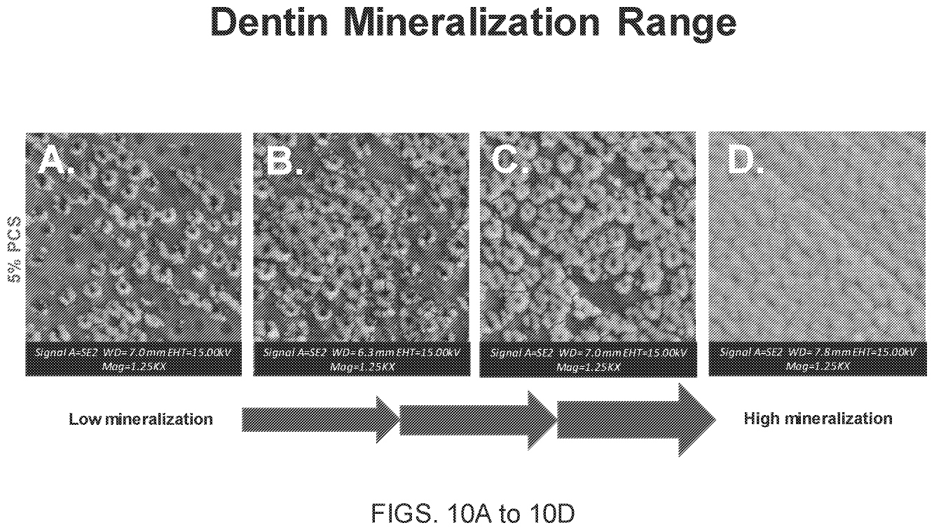

In order to assess patterns of mineralization, different regions of a CaP sheet formed after treatment with 5% PCS were evaluated under 1.25 kX magnification. The resulting images, shown in FIGS. 10A to 10D indicated remineralization seems to be initiated within the dentinal tubules and continue to extend from these locations until a sheet is formed. This result provides support for the use of PCS in the treatment of dentin hypersensitivity.

What is claimed:

1. A method for remineralizing tooth dentin comprising contacting the tooth dentin with a catechol-containing material comprising one or more of catechol, semi-quinone, or quinone.

2. The method of claim 1, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of: a) a reactive species separate from the catechol or catechol containing material; and b) a catalyst, co-catalyst or an accelerator.

3. The method of claim 2, wherein the catechol-containing material comprises the reactive species separate from the catechol or catechol containing material; and the reactive species comprises an acrylic, a silane, a silicone, a methacrylate, a polyvinyl alcohol (PVA), a urethane, or a combination thereof.

4. The method of claim 3, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

5. The method of claim 1 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

6. The method of claim 3, wherein the reactive species is the acrylate.

7. The method of claim 1, wherein the catechol-containing material is disposed upon a tooth dentin.

8. The method of claim 1, wherein the catechol-containing has a thickness of from about 10 nanometers to about 500 microns.

9. The method of claim 1, wherein the catechol-containing material comprises about 0.1% poly(catechol-styrene) (PCS).

10. The method of claim 1, wherein the catechol-containing material comprises an amount of PCS in the range of from about 0.1% to about 10%.

11. The method of claim 1, wherein the catechol-containing material comprises at least partially oxidized PCS.

12. The method of claim 1, wherein the catechol-containing material comprises at least partially reduced PCS.

13. The method of claim 1, wherein the catechol-containing material comprises material having at least one partially intermediate oxidation state.

14. A method for treating dental hypersensitivity in a subject comprising contacting exposed dentin tubules with a catechol-containing material comprising catechol, semi-quinone, or quinone.

15. The method of claim 14, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of:
a) a reactive species separate from the catechol or catechol containing material; and b) a catalyst, co-catalyst or an accelerator.

16. The method of claim 15, wherein the catechol-containing material comprises the reactive species separate from the catechol or catechol containing material; and the reactive species comprises an acrylic, a silane, a silicone, a methacrylate, a polyvinyl alcohol (PVA) or a combination thereof.

17. The method of claim 16, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

18. The method of claim 14 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

19. The method of claim 16, wherein the reactive species is the acrylate.

20. The method of claim 14, wherein the catechol-containing material is disposed upon a tooth dentin.

21. The method of claim 14, wherein the catechol-containing has a thickness of from about 10 nanometers to about 500 microns.

22. The method of claim 14, wherein the catechol-containing material comprises about 0.1% poly(catechol-styrene) (PCS).

23. The method of claim 14, wherein the catechol-containing material comprises an amount of PCS in the range of from about 0.1% to about 10%.

24. The method of claim 14, wherein the catechol-containing material comprises at least partially oxidized PCS.

25. The method of claim 14, wherein the catechol-containing material comprises at least partially reduced PCS.

26. The method of claim 14, wherein the catechol-containing material comprises material having at least one partially intermediate oxidation state.

27. A method for restoring acid-etched tooth dentin in a subject comprising contacting the tooth dentin with a catechol-containing material comprising catechol, semi-quinone, or quinone.

28. The method of claim 27, wherein the catechol-containing material comprises monomeric, oligomeric, or polymeric catechol or catechol containing material, wherein said catechol presents as a catechol and/or as a semi-quinone and/or as a quinone without the presence of an amine; and wherein the polymeric layer optionally comprises at least one of:
a) a reactive species separate from the catechol or catechol containing material; and b) a catalyst, co-catalyst or an accelerator.

29. The method of claim 28, wherein the catechol-containing material comprises the reactive species separate from the catechol or catechol containing material; and the reactive species comprises an acrylic, a silane, a silicone, a methacrylate, a polyvinyl alcohol (PVA) or a combination thereof.

30. The method of claim 29, wherein the catechol-containing material comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane dimethacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

31. The method of claim 27 further comprising free radical polymerization initiators such as acrylate polymerization initiators, including those that are light activated, such as benzoyl peroxide (BPO), 2,3-bornanedione (Camphorquinone), Ethyl-4-(dimethylamino)benzoate (EDMAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

32. The method of claim 29, wherein the reactive species is the acrylate.

33. The method of claim 27, wherein the catechol-containing material is disposed upon a tooth dentin.

34. The method of claim 27, wherein the catechol-containing has a thickness of from about 10 nanometers to about 500 microns.

35. The method of claim 27, wherein the catechol-containing material comprises about 0.1% poly(catechol-styrene) (PCS).

36. The method of claim 27, wherein the catechol-containing material comprises an amount of PCS in the range of from about 0.1% to about 10%.

37. The method of claim 27, wherein the catechol-containing material comprises at least partially oxidized PCS.

38. The method of claim 27, wherein the catechol-containing material comprises at least partially reduced PCS.

39. The method of claim 27, wherein the catechol-containing material comprises material having at least one partially intermediate oxidation state.
